(19) United States
(12) Patent Application Publication
(10) Pub. No.: US 2006/0211676 A1
(43) Pub. Date: Sep. 21, 2006

(54) Crystalline anhydrous cefdinir and crystalline cefdinir hydrates

(76) Inventors: Devalina Law, Libertyville, IL (US); Rodger F. Henry, Wildwood, IL (US); Xiaochun Lou, Long Grove, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/221,427

(22) Filed: Sep. 8, 2005

Related U.S. Application Data
(63) Continuation-in-part of application No. 11/177,640, filed on Jul. 8, 2005.
Application No. 11/177,640 is a continuation-in-part of application No. 11/072,568, filed on Mar. 3, 2005.
(60) Provisional application No. 60/553,643, filed on Mar. 16, 2004.

(51) Int. Cl. A61K 31/545; C07D 501/00

(52) U.S. Cl. 514/200; 540/222

(57) ABSTRACT

A novel crystalline cefdinir anhydrate and novel crystalline cefdinir hydrates, ways to make them and use them, compositions comprising them and made with them, and methods of treatment using them are disclosed.

This application is a continuation-in-part of U.S. Ser. No. 11/177,640 filed on Jul. 8, 2005, which is a continuation in part of U.S. Ser. No. 11/072,568 filed on Mar. 3, 2005, which claims priority from U.S. Provisional Ser. No. 60/553,643 filed on Mar. 16, 2004, the contents of which are incorporated herein by reference.

### FIELD OF THE INVENTION

This invention pertains to a crystalline cefdinir anhydrate and crystalline cefdinir hydrates, ways to make them and use them, compositions comprising them and made with them, and methods of treatment using them.

### BACKGROUND OF THE INVENTION

Cefdinir, marketed in the United States as OMNICEF®, is an antibiotic available as capsules or particles for suspension. Cefdinir is useful for treating infections resulting from bacteria such as Staphylococcus aureus, Streptococcus pneumoniae, Streptococcus pyogenes, Hemophilus influenzae, Moraxella catarrhalis, E. coli, Klebsiella and Proteus mirabilis.

Crystallinity of compounds may effect, among other physical and mechanical properties, their solubility, dissolution rate, hardness, compressability and melting point. Because the ease of manufacture and use of cefdinir is dependent on its solubility, dissolution rate and melting point, there is an existing need in the chemical and therapeutic arts for identification of novel crystalline forms of cefdinir and ways to reproducibly make them.

### BRIEF DESCRIPTION OF THE FIGURES

FIG. 1 shows a picture of the unit cell of cefdinir trihemihydrate.

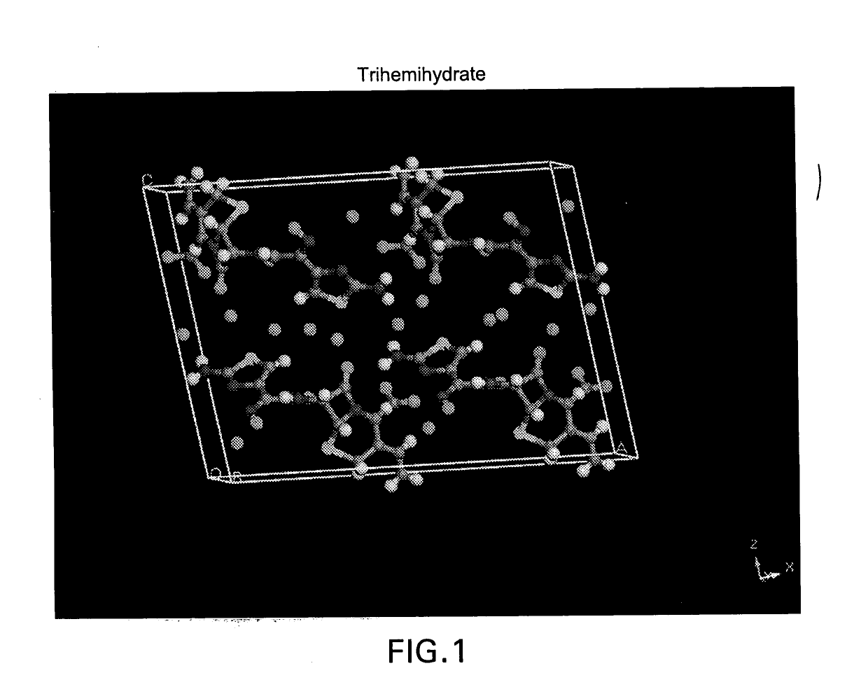

FIG. 2 shows an experimental and calculated powder X-ray diffraction pattern of cefdinir trihemihydrate.

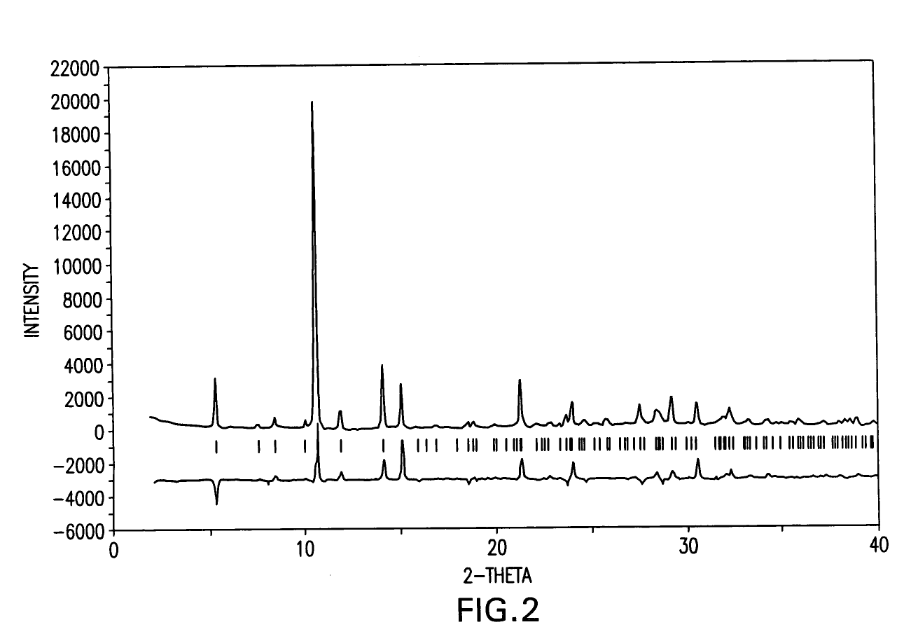

FIG. 3 shows a picture of the unit cell of cefdinir sesquihydrate.

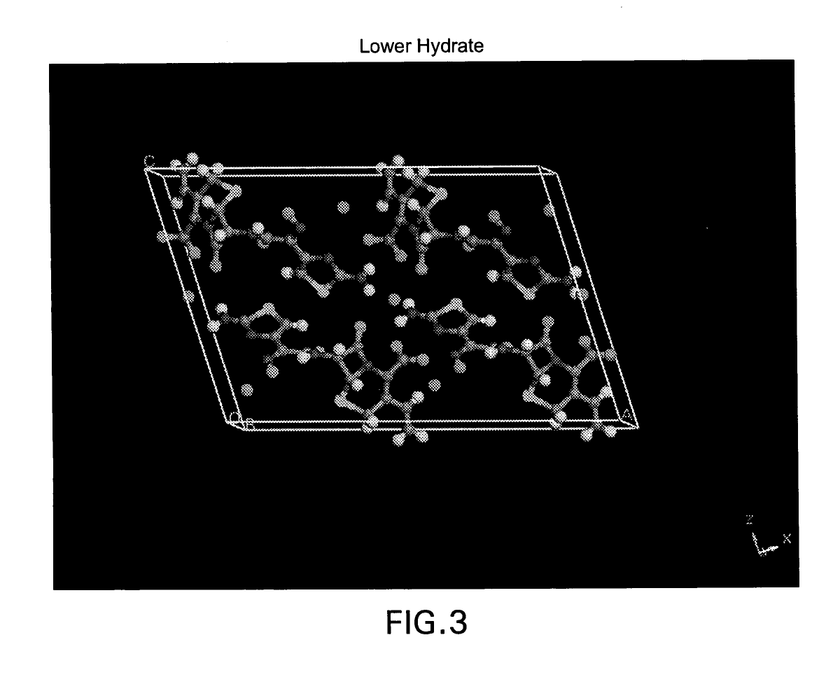

FIG. 4 shows the powder X-ray diffraction pattern of cefdinir sesquihydrate.

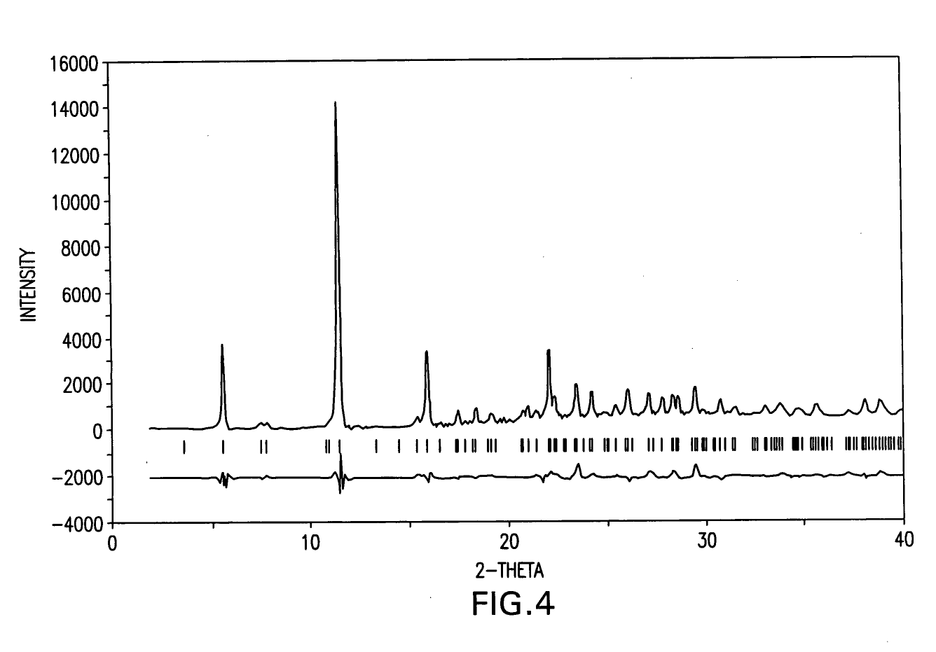

FIG. 5 shows the powder X-ray diffraction pattern of cefdinir anhydrate.

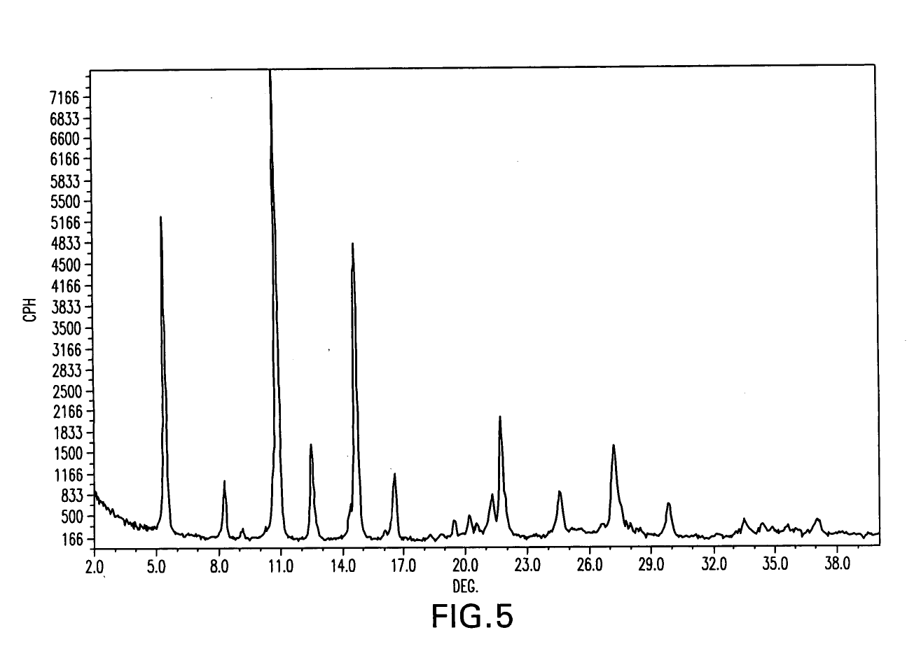

FIG. 6 shows two powder X-ray diffraction patterns of cefdinir.1.5H₂O (about 6% water) and cefdinir.H₂O (about 4% water).

FIG. 7 shows the Dynamic Moisture Sorption/Desorption Gravimetric analysis showing the desorption isotherm of Cefdinir hydrates.

FIG. 8 shows the results of a thermogravimetric analysis (TGA) of the conversion of cefdinir trihemihydrate to cefdinir sesquihydrate to the cefdinir anhydrate of this invention.

### SUMMARY OF THE INVENTION

One embodiment of this invention pertains to a novel crystalline cefdinir anhydrate, said novel crystalline cefdinir anhydrate characterized, when measured with radiation at 1.54178 Å, comprising a powder diffraction pattern having 2θ values of about 5.5°, 10.9°, 12.6°, 14.7°, 16.6°, 21.8° and 27.3°.

Another embodiment pertains to pharmaceutical compositions comprising a novel crystalline cefdinir anhydrate, said novel crystalline cefdinir anhydrate characterized, when measured with radiation at 1.54178 Å, comprising a powder diffraction pattern having 2θ values of about 5.5°, 10.9°, 12.6°, 14.7°, 16.6°, 21.8° and 27.3°.

Still another embodiment pertains to methods of treating bacterial infection in a mammal, particularly in humans, comprising administering thereto a therapeutically effective amount of a novel crystalline cefdinir anhydrate, said novel crystalline cefdinir anhydrate characterized, when measured with radiation at 1.54178 Å, comprising a powder diffraction pattern having 2θ values of about 5.5°, 10.9°, 12.6°, 14.7°, 16.6°, 21.8° and 27.3°.

Still another embodiment pertains to novel isolated crystalline cefdinir lower hydrates and novel iso-structural hydrates thereof, said novel isolated crystalline cefdinir lower hydrates and said novel iso-structural hydrates thereof characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to pharmaceutical compositions comprising a novel isolated crystalline cefdinir lower hydrate or a novel isolated iso-structural hydrate thereof, said novel isolated crystalline cefdinir lower hydrate and said novel isolated iso-structural hydrate thereof characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02 or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to methods of treating bacterial infection in a mammal, particularly in humans, comprising administering thereto a therapeutically effective amount of a novel isolated crystalline cefdinir lower hydrate or a novel isolated iso-structural hydrate thereof, said novel isolated crystalline cefdinir lower hydrate and said novel isolated iso-structural hydrate thereof characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02°, or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated crystalline cefdinir trihemihydrate, with or without surface water, said novel isolated crystalline cefdinir trihemihydrate characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

Still another embodiment pertains to pharmaceutical compositions comprising a novel isolated crystalline cefdinir trihemihydrate, with or without surface water, said novel isolated crystalline cefdinir trihemihydrate characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1°, and 30.5° or by a combination thereof.

Still another embodiment pertains to methods of treating bacterial infection in a mammal, particularly in humans, comprising administering thereto a therapeutically effective amount of novel isolated crystalline cefdinir trihemihydrate, with or without surface water, said novel isolated crystalline cefdinir trihemihydrate characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5° or by a combination thereof.

### DETAILED DESCRIPTION OF THE INVENTION

One embodiment of this invention pertains to a novel crystalline cefdinir anhydrate, said novel crystalline cefdinir anhydrate characterized, when measured with radiation at 1.54178 Å, comprising a powder diffraction pattern having 2θ values of about 5.5°, 10.9°, 12.6°, 14.7°, 16.6°, 21.8° and 27.3°.

Another embodiment pertains to a novel crystalline cefdinir anhydrate having substantial crystalline purity, said novel crystalline cefdinir anhydrate characterized, when measured with radiation at 1.54178 Å, comprising a powder diffraction pattern having 2θ values of about 5.5°, 10.9°, 12.6°, 14.7°, 16.6°, 21.8° and 27.3°.

Still another embodiment pertains to a novel crystalline cefdinir anhydrate having substantial crystalline purity and substantial chemical purity, said novel crystalline cefdinir anhydrate characterized, when measured with radiation at 1.54178 Å, comprising a powder diffraction pattern having 2θ values of about 5.5°, 10.9°, 12.6°, 14.7°, 16.6°, 21.8° and 27.3°.

Still another embodiment pertains to a novel crystalline cefdinir anhydrate having substantial crystalline purity, substantial chemical purity, and substantial isomeric purity, said novel crystalline cefdinir anhydrate characterized, when measured with radiation at 1.54178 Å, comprising a powder diffraction pattern having 2θ values of about 5.5°, 10.9°, 12.6°, 14.7°, 16.6°, 21.8° and 27.3°.

Still another embodiment pertains to pharmaceutical compositions comprising a novel crystalline cefdinir anhydrate, said novel crystalline cefdinir anhydrate characterized, when measured with radiation at 1.54178 Å, comprising a powder diffraction pattern having 2θ values of about 5.5°, 10.9°, 12.6°, 14.7°, 16.6°, 21.8° and 27.3°.

Still another embodiment pertains to methods of treating bacterial infection in a mammal, particularly in humans, comprising administering thereto a therapeutically effective amount of a novel crystalline cefdinir anhydrate, said novel crystalline cefdinir anhydrate characterized, when measured with radiation at 1.54178 Å, comprising a powder diffraction pattern having 2θ values of about 5.5°, 10.9°, 12.6°, 14.7°, 16.6°, 21.8° and 27.3°.

Still another embodiment pertains to novel isolated crystalline cefdinir lower hydrates and novel iso-structural hydrates thereof, said novel isolated crystalline cefdinir lower hydrates and said novel iso-structural hydrates thereof characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated crystalline cefdinir lower hydrates and novel iso-structural hydrates thereof having substantial crystalline purity, said novel isolated crystalline cefdinir lower hydrates and said novel iso-structural hydrates thereof characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated crystalline cefdinir lower hydrates and novel isolated iso-structural hydrates thereof having substantial crystalline purity and substantial chemical purity, said novel isolated crystalline cefdinir lower hydrates and said novel isolated iso-structural hydrates thereof characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated crystalline cefdinir lower hydrates and novel isolated iso-structural hydrates thereof having substantial crystalline purity, substantial chemical purity and substantial isomeric purity, said novel isolated crystalline cefdinir lower hydrates and said novel isolated iso-structural hydrates thereof characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to pharmaceutical compositions comprising a novel isolated crystalline cefdinir lower hydrate or a novel isolated iso-structural hydrate thereof, said novel isolated crystalline cefdinir lower hydrate and said novel isolated iso-structural hydrate thereof characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to methods of treating bacterial infection in a mammal particularly in humans, comprising administering thereto a therapeutically effective amount of a novel isolated crystalline cefdinir lower hydrate or a novel isolated iso-structural hydrate thereof, said novel isolated crystalline cefdinir lower hydrate and said novel isolated iso-structural hydrate thereof characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated iso-structural hydrates of a crystalline cefdinir lower hydrate, said novel isolated iso-structural hydrates of said crystalline cefdinir lower hydrates characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated iso-structural hydrates of a crystalline cefdinir lower hydrate having substantial crystalline purity, said isolated novel isolated iso-structural hydrates characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated iso-structural hydrates of a crystalline cefdinir lower hydrate having substantial crystalline purity and substantial chemical purity, said novel isolated iso-structural hydrates characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated iso-structural hydrates of a crystalline cefdinir lower hydrate having substantial crystalline purity, substantial chemical purity and substantial isomeric purity, said novel isolated iso-structural hydrates characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to pharmaceutical compositions comprising a novel isolated iso-structural hydrate of crystalline cefdinir lower hydrate, said novel isolated iso-structural hydrate of said crystalline cefdinir lower hydrate characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to methods of treating bacterial infection in a mammal particularly in humans, comprising administering thereto a therapeutically effective amount of a novel isolated iso-structural hydrate of a crystalline cefdinir lower hydrate, said novel isolated iso-structural hydrate of said crystalline cefdinir lower hydrate characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated crystalline cefdinir.0.87H₂O, said novel isolated crystalline cefdinir.0.87H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated crystalline cefdinir.0.87H₂O having substantial crystalline purity, said novel isolated crystalline cefdinir.0.87H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated crystalline cefdinir.0.87H₂O having substantial crystalline purity and substantial chemical purity, said novel isolated crystalline cefdinir.0.87H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated crystalline cefdinir.0.87H₂O having substantial crystalline purity, substantial chemical purity and substantial isomeric purity, said novel isolated crystalline cefdinir.0.87H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to pharmaceutical compositions comprising a novel isolated crystalline cefdinir.0.87H₂O, said novel isolated crystalline cefdinir.0.87H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to methods of treating bacterial infection in a mammal, particularly in humans, comprising administering thereto a therapeutically effective amount of novel isolated crystalline cefdinir.0.87H₂O, said novel isolated crystalline cefdinir.0.87H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated crystalline cefdinir.1.14H₂O, said novel isolated crystalline cefdinir.1.14H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated crystalline cefdinir.1.14H₂O having substantial crystalline purity, said novel isolated crystalline cefdinir.1.14H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated crystalline cefdinir.1.14H₂O having substantial crystalline purity and substantial chemical purity, said novel isolated crystalline cefdinir.1.14H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated crystalline cefdinir.1.14H₂O having substantial crystalline purity, substantial chemical purity and substantial isomeric purity, said novel isolated crystalline cefdinir.1.14H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to pharmaceutical compositions comprising a novel isolated crystalline cefdinir.1.14H₂O, said novel isolated crystalline cefdinir.1.14H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to methods of treating bacterial infection in a mammal, particularly in humans, comprising administering thereto a therapeutically effective amount of novel isolated cefdinir.1.14H₂O, said novel isolated crystalline cefdinir.1.14H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated crystalline cefdinir.1.33H₂O, said novel isolated crystalline cefdinir.1.33H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated crystalline cefdinir.1.33H₂O having substantial crystalline purity, said novel isolated crystalline cefdinir.1.33H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated crystalline cefdinir.1.33H₂O having substantial crystalline purity and substantial chemical purity, said novel isolated crystalline cefdinir.1.33H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated crystalline cefdinir.1.33H₂O having substantial crystalline purity, substantial chemical purity and substantial isomeric purity, said novel isolated crystalline cefdinir.1.33H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to pharmaceutical compositions comprising a novel isolated crystalline cefdinir.1.33H₂O, said novel isolated crystalline cefdinir.1.33H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to methods of treating bacterial infection in a mammal, particularly in humans, comprising administering thereto a therapeutically effective amount of novel isolated crystalline cefdinir.1.33H₂O, said novel isolated crystalline cefdinir.1.33H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated crystalline cefdinir.1.43H₂O, said novel isolated crystalline cefdinir.1.43H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated crystalline cefdinir.1.43H₂O having substantial crystalline purity, said novel isolated crystalline cefdinir.1.43H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated crystalline cefdinir.1.43H₂O having substantial crystalline purity and substantial chemical purity, said novel isolated crystalline cefdinir.1.43H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated crystalline cefdinir.1.43H₂O having substantial crystalline purity, substantial chemical purity and substantial isomeric purity, said novel isolated crystalline cefdinir.1.43H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to pharmaceutical compositions comprising a novel isolated crystalline cefdinir.1.43H₂O, said novel isolated crystalline cefdinir.1.43H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to methods of treating bacterial infection in a mammal, particularly in humans, comprising administering thereto a therapeutically effective amount of novel isolated cefdinir.1.43H₂O, said novel isolated crystalline cefdinir.1.43H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to novel isolated crystalline cefdinir trihemihydrate, with or without surface water, said novel isolated crystalline cefdinir trihemihydrate characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1°, and 30.5°, or by a combination thereof.

Still another embodiment pertains to novel isolated crystalline cefdinir trihemihydrate, with or without surface water, said novel isolated crystalline cefdinir trihemihydrate having substantial crystalline purity, said isolated crystalline cefdinir trihemihydrate characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1°, and 30.5°, or by a combination thereof.

Still another embodiment pertains to novel isolated crystalline cefdinir trihemihydrate, with or without surface water, having substantial crystalline purity and substantial chemical purity, said novel isolated crystalline cefdinir trihemihydrate characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

Still another embodiment pertains to novel isolated crystalline cefdinir trihemihydrate, with or without surface water, having substantial crystalline purity, substantial chemical purity and substantial isomeric purity, said novel isolated cefdinir trihemihydrate characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

Still another embodiment pertains to pharmaceutical compositions comprising a novel isolated crystalline cefdinir trihemihydrate, with or without surface water, said novel isolated crystalline cefdinir trihemihydrate characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1°, and 30.5°, or by a combination thereof.

Still another embodiment pertains to methods of treating bacterial infection in a mammal, particularly in humans, comprising administering thereto a therapeutically effective amount of novel isolated crystalline cefdinir trihemihydrate, with or without surface water, said novel isolated crystalline cefdinir trihemihydrate characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1°, and 30.5°, or by a combination thereof.

Still another embodiment pertains to mixtures, including isolated mixtures and non-isolated mixtures, comprising Cefdinir Crystalline Form A and a novel crystalline cefdinir anhydrate, said novel crystalline cefdinir anhydrate characterized, when measured with radiation at 1.54178 Å, comprising a powder diffraction pattern having 2θ values of about 5.5°, 10.9°, 12.6°, 14.7°, 16.6°, 21.8° and 27.3°.

Still another embodiment pertains to pharmaceutical compositions comprising Cefdinir Crystalline Form A and a novel crystalline cefdinir anhydrate, said novel crystalline cefdinir anhydrate characterized, when measured with radiation at 1.54178 Å, comprising a powder diffraction pattern having 2θ values of about 5.5°, 10.9°, 12.6°, 14.7°, 16.6°, 21.8° and 27.3°.

Still another embodiment pertains to methods of treating bacterial infection in a mammal, particularly in humans, comprising administering thereto a therapeutically effective amount of a mixture of Cefdinir Crystalline Form A and a novel crystalline cefdinir anhydrate, said novel crystalline cefdinir anhydrate characterized, when measured with radiation at 1.54178 Å, comprising a powder diffraction pattern having 2θ values of about 5.5°, 10.9°, 12.6°, 14.7°, 16.6°, 21.8° and 27.3°.

Still another embodiment pertains to mixtures, including isolated mixtures and non-isolated mixtures, comprising Cefdinir Crystalline Form A and a novel iso-structural hydrate of a crystalline cefdinir lower hydrate, said novel iso-structural hydrate of said crystalline cefdinir lower hydrate characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to pharmaceutical compositions comprising Cefdinir Crystalline Form A and a novel iso-structural hydrate of a crystalline cefdinir lower hydrate, said novel iso-structural hydrate of said crystalline cefdinir lower hydrate characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to methods of treating bacterial infection in a mammal, particularly in humans, comprising administering thereto a therapeutically effective amount of a mixture of Cefdinir Crystalline Form A and a novel iso-structural hydrate of a crystalline cefdinir lower hydrate, said novel iso-structural hydrate of said crystalline cefdinir lower hydrate characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to mixtures, including isolated mixtures and non-isolated mixtures, comprising Cefdinir Crystalline Form A and novel crystalline cefdinir.0.87H₂O, said novel cefdinir 0.87H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to pharmaceutical compositions comprising Cefdinir Crystalline Form A and novel cefdinir.0.87H₂O, said novel cefdinir.0.87H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to methods of treating bacterial infection in a mammal, particularly in humans, comprising administering thereto a therapeutically effective amount of a mixture of Cefdinir Crystalline Form A and novel cefdinir.0.87H₂O, said novel cefdinir.0.87H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to mixtures, including isolated mixtures and non-isolated mixtures, comprising Cefdinir Crystalline Form A and novel crystalline cefdinir.1.14H₂O, said novel cefdinir.1.14H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to pharmaceutical compositions comprising Cefdinir Crystalline Form A and novel cefdinir.1.14H₂O, said novel cefdinir.1.14H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to methods of treating bacterial infection in a mammal, particularly in humans, comprising administering thereto a therapeutically effective amount of a mixture of Cefdinir Crystalline Form A and novel cefdinir.1.14H₂O, said novel cefdinir.1.14H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to mixtures, including isolated mixtures and non-isolated mixtures, comprising Cefdinir Crystalline Form A and novel crystalline cefdinir.1.33H₂O, said novel cefdinir.1.33H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to pharmaceutical compositions comprising Cefdinir Crystalline Form A and novel cefdinir.1.33H₂O, said novel cefdinir.1.33H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23. 10, or by a combination thereof.

Still another embodiment pertains to methods of treating bacterial infection in a mammal, particularly in humans, comprising administering thereto a therapeutically effective amount of a mixture of Cefdinir Crystalline Form A and novel cefdinir.1.33H₂O, said novel cefdinir.1.33H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to mixtures, including isolated mixtures and non-isolated mixtures, comprising Cefdinir Crystalline Form A and novel crystalline cefdinir.1.43H₂O, said novel cefdinir.1.43H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to pharmaceutical compositions comprising Cefdinir Crystalline Form A and novel cefdinir.1.43H₂O, said novel cefdinir.1.43H₂O characterized, in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to methods of treating bacterial infection in a mammal, particularly in humans, comprising administering thereto a therapeutically effective amount of a mixture of Cefdinir Crystalline Form A and novel cefdinir.1.43H₂O, said novel cefdinir.1.43H₂O characterized, in the monoclinic crystal system and C2 space group when measured with radiation at 0.7107 Å, by-respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9°, 8.1°, 11.8°, 15.7°, 16.2°, 22.6° and 23.1°, or by a combination thereof.

Still another embodiment pertains to mixtures, including isolated mixtures and non-isolated mixtures, comprising Cefdinir Crystalline Form A and a novel crystalline cefdinir trihemihydrate, with or without surface water, said novel crystalline cefdinir trihemihydrate characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1°, and 30.5° or by a combination thereof.

Still another embodiment pertains to pharmaceutical compositions comprising Cefdinir Crystalline Form A and a novel crystalline cefdinir trihemihydrate, with or without surface water, said novel crystalline cefdinir trihemihydrate characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1°, and 30.5°, or by a combination thereof.

Still another embodiment pertains to methods of treating bacterial infection in a mammal, particularly in humans, comprising administering thereto a therapeutically effective amount of a mixture of Cefdinir Crystalline Form A and a novel crystalline cefdinir trihemihydrate, with or without surface water, said novel crystalline cefdinir trihemihydrate characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1°, and 30.5°, or by a combination thereof.

Still another embodiment pertains to mixtures, including isolated mixtures and non-isolated mixtures, comprising Cefdinir Crystalline Form A and novel crystalline cefdinir.3.73H₂O, said novel crystalline cefdinir.3.73H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1°, and 30.5°, or by a combination thereof.

Still another embodiment pertains to pharmaceutical compositions comprising Cefdinir Crystalline Form A and novel crystalline cefdinir.3.73H₂O, said novel crystalline cefdinir.3.73H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

Still another embodiment pertains to methods of treating bacterial infection in a mammal, particularly in humans, comprising administering thereto a therapeutically effective amount of a mixture of Cefdinir Crystalline Form A and novel crystalline cefdinir.3.73H₂O, said novel crystalline cefdinir.3.73H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1°, and 30.5°, or by a combination thereof.

Still another embodiment pertains to mixtures, including isolated mixtures and non-isolated mixtures, comprising Cefdinir Crystalline Form A and novel crystalline cefdinir.3.76H₂O, said novel crystalline cefdinir.3.76H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1°, and 30.5°, or bya combination thereof.

Still another embodiment pertains to pharmaceutical compositions comprising Cefdinir Crystalline Form A and novel crystalline cefdinir.3.76H₂O, said novel crystalline cefdinir.3.76H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02 or, when measured with radiation at 1.54178 Å, comprising a powder diffraction pattern having 2θ values of about 5.3°, 10.6°, 14.1°, 15.1°, 21.3°, 29.1°, and 30.5°, or by a combination thereof.

Still another embodiment pertains to methods of treating bacterial infection in a mammal, particularly in humans, comprising administering thereto a therapeutically effective amount of a mixture of Cefdinir Crystalline Form A and novel crystalline cefdinir.3.76H₂O, said novel crystalline cefdinir.3.76H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1°, and 30.5°, or by a combination thereof.

Still another embodiment pertains to mixtures, including isolated mixtures and non-isolated mixtures, comprising Cefdinir Crystalline Form A and novel crystalline cefdinir.3.79H₂O, said novel crystalline cefdinir.3.79H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1°, and 30.5°, or by a combination thereof.

Still another embodiment pertains to pharmaceutical compositions comprising Cefdinir Crystalline Form A and novel crystalline cefdinir.3.79H₂O, said novel crystalline cefdinir.3.79H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1°, and 30.5°, or by a combination thereof.

Still another embodiment pertains to methods of treating bacterial infection in a mammal, particularly in humans, comprising administering thereto a therapeutically effective amount of a mixture of Cefdinir Crystalline Form A and novel crystalline cefdinir.3.79H₂O, said novel crystalline cefdinir.3.79H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1°, and 30.5°, or by a combination thereof.

Still another embodiment pertains to mixtures, including isolated mixtures and non-isolated mixtures, comprising Cefdinir Crystalline Form A and novel crystalline cefdinir.3.81H₂O, said novel crystalline cefdinir.3.81H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1°, and 30.5°, or by a combination thereof.

Still another embodiment pertains to pharmaceutical compositions comprising Cefdinir Crystalline Form A and novel crystalline cefdinir.3.81H₂O, said novel crystalline cefdinir.3.81H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

Still another embodiment pertains to methods of treating bacterial infection in a mammal, particularly in humans, comprising administering thereto a therapeutically effective amount of a mixture of Cefdinir Crystalline Form A and novel crystalline cefdinir.3.81H₂O, said novel crystalline cefdinir.3.81H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1°, and 30.5°, or by a combination thereof.

Still another embodiment pertains to a process for making crystalline cefdinir trihemihydrate, with or without surface water, said process comprising:

providing a mixture comprising cefdinir and solvent;

causing crystalline cefdinir trihemihydrate to exist in said mixture, said crystalline cefdinir trihemihydrate, when isolated with or without surface water, and characterized in the monoclinic crystal system and C2 space group with radiation at 0.7107 Å, by respective lattice parameters a, band c of 23.77 Å±0.02 Å, 5.007 Å±0.004 Å and 16.76 Å±0.01 Å and β of 100.29°±0.02°; and

isolating said crystalline cefdinir trihemihydrate.

Crystalline cefdinir trihemihydrate, with or without surface water, prepared by a process comprising providing a mixture comprising cefdinir and solvent, causing crystalline cefdinir trihemihydrate to exist in said mixture, said crystalline cefdinir trihemihydrate, when isolated with or without surface water and characterized in the monoclinic crystal system and C2 space group with radiation at 0.7107 Å, by respective lattice parameters a, b and c of 23.77 Å±0.02 Å, 5.007 Å±0.004 Å and 16.76 Å±0.01 Å and β of 100.29°±0.02°; and isolating said crystalline cefdinir trihernihydrate.

Still another embodiment pertains to a process for making crystalline cefdinir trihemihydrate, with or without surface water, said process comprising:

providing a mixture comprising semisolid cefdinir, ethyl acetate, ethanol and acid or base;

adding water to said mixture to form crystalline cefdinir trihemihydrate, said crystalline cefdinir trihemihydrate, when isolated with or without surface water and characterized with radiation at 0.7107 Å in the monoclinic crystal system and C2 space group, by respective lattice parameters a, b and c of 23.77 Å±0.02 Å, 5.007 Å±0.004 Å and 16.76 Å±0.01 Å and β of 100.29°±0.02°; and

isolating said crystalline cefdinir trihemihydrate.

Still another embodiment pertains to crystalline cefdinir trihemihydrate, with or without surface water, prepared by a process comprising providing a mixture comprising semisolid cefdinir, ethyl acetate, ethanol and acid or base, adding water to said mixture to form crystalline cefdinir trihemihydrate, said crystalline cefdinir trihemihydrate, when isolated with or without surface water and characterized with radiation at 0.7107 Å in the monoclinic crystal system and C2 space group, by respective lattice parameters a, b and c of 23.77 Å±0.02 Å, 5.007 Å±0.004 Å and 16.76 Å±0.01 Å and β of 100.29°±0.02°; and isolating said crystalline cefdinir trihemihydrate.

Still another embodiment pertains to a process for making crystalline cefdinir trihemihydrate with or without surface water comprising converting 7-amino-3-vinyl-3-cephem-4-carboxylic acid ((6R,7R)-7-amino-8-oxo-3-vinyl-5-thia-1-azabicyclo[4.2.0]oct-2-ene-2-carboxylic acid) to crystalline cefdinir trihemihydrate, with or without carboxylic acid protection and deprotection steps, said process further comprising converting semisolid cefdinir containing at least one residual solvent from said process to crystalline cefdinir trihemihydrate.

Still another embodiment pertains to crystalline cefdinir trihemihydrate prepared by a process comprising converting 7-amino-3-vinyl-3-cephem-4-carboxylic acid ((6R,7R)-7-amino-8-oxo-3-vinyl-5-thia-1-azabicyclo[4.2.0]oct-2-ene-2-carboxylic acid) to crystalline cefdinir trihemihydrate, with or without carboxylic acid protection and deprotection steps, and further comprising converting semisolid cefdinir containing at least one residual solvent from said process to crystalline cefdinir trihemihydrate.

Still another embodiment pertains to a process for making crystalline cefdinir trihemihydrate comprising making a mixture of semisolid cefdinir and solvent and converting said semisolid cefdinir to crystalline cefdinir trihemihydrate.

Still another embodiment pertains to crystalline cefdinir trihemihydrate prepared by a process comprising making a mixture of semisolid cefdinir and solvent and converting semisolid cefdinir to crystalline cefdinir trihemihydrate.

Still another embodiment pertains to a process for making crystalline cefdinir trihemihydrate comprising making a mixture of semisolid cefdinir and solvent and adding water to said mixture.

Still another embodiment pertains to crystalline cefdinir trihemihydrate prepared by a process comprising making a mixture of semisolid cefdinir and solvent and adding water to said mixture.

Still another embodiment pertains to a process for converting semisolid cefdinir to crystalline cefdinir trihemihydrate comprising making a mixture of cefdinir semisolid and solvent, adding water to said mixture and isolating said crystalline cefdinir trihemihydrate.

Still another embodiment pertains to crystalline cefdinir trihemihydrate prepared by a process comprising making a mixture of cefdinir semisolid and solvent, adding water to said mixture and isolating said crystalline cefdinir trihemihydrate.

Still another embodiment pertains to a process for converting semisolid cefdinir to crystalline cefdinir trihemihydrate comprising making a mixture of cefdinir semisolid and solvent, adding water to said mixture, centrifugating said mixture and decanting said solvent.

Still another embodiment pertains to crystalline cefdinir trihemihydrate prepared by a process comprising making a mixture of cefdinir semisolid and solvent, adding water to said mixture, centrifugating said mixture and decanting said solvent.

Still another embodiment pertains to a process for converting semisolid cefdinir to crystalline cefdinir trihemihydrate comprising making a mixture of cefdinir semisolid and solvent, adding water to said mixture, centrifugating said mixture and filtering said mixture under positive pressure.

Still another embodiment pertains to crystalline cefdinir trihemihydrate prepared by a process comprising making a mixture of cefdinir semisolid and solvent, adding water to said mixture, centrifugating said mixture and filtering said mixture under positive pressure.

Still another embodiment pertains to a process for converting crystalline cefdinir trihemihydrate, with or without surface water, to a crystalline cefdinir lower hydrate, or an iso-structural hydrate thereof, by heating at about 25° C. to about 70° C.

Still another embodiment pertains to a crystalline cefdinir lower hydrate, or an iso-structural hydrate thereof, prepared by heating crystalline cefdinir trihemihydrate, with or without surface water, at about 25° C. to about 70° C.

Still another embodiment pertains to a process for converting crystalline cefdinir trihemihydrate, with or without surface water, to a crystalline cefdinir lower hydrate, or an iso-structural hydrate thereof, by heating at about 25° C. to about 70° C. for about 30 minutes to about 24 hours.

Still another embodiment pertains to a crystalline cefdinir lower hydrate, or an iso-structural hydrate thereof, prepared by heating crystalline cefdinir trihemihydrate at about 25° C. to about 70° C. for about 30 minutes to about 24 hours.

Still another embodiment pertains to a process for converting a cefdinir lower hydrate, or an iso-structural hydrate thereof, to Cefdinir Crystalline Form A by providing a mixture comprising a cefdinir lower hydrate or an iso-structural hydrate thereof and an alcohol in which said cefdinir lower hydrate or said iso-structural hydrate thereof completely dissolves, causing Cefdinir Crystalline Form A to exist in said mixture, and isolating said Cefdinir Crystalline Form A.

Still another embodiment pertains to Cefdinir Crystalline Form A prepared by a process comprising providing a mixture comprising a cefdinir lower hydrate or an iso-structural hydrate thereof and an alcohol in which said cefdinir lower hydrate or said iso-structural hydrate thereof completely dissolves, causing Cefdinir Crystalline Form A to exist in said mixture, and isolating said Cefdinir Crystalline Form A.

Still another embodiment pertains to a process for converting cefdinir.0.87H₂O, to Cefdinir Crystalline Form A by providing a mixture comprising cefdinir.0.87H₂O and an alcohol in which said cefdinir.0.87H₂O completely dissolves, causing Cefdinir Crystalline Form A to exist in said mixture, and isolating said Cefdinir Crystalline Form A.

Still another embodiment pertains to Cefdinir Crystalline Form A prepared by a process comprising providing a mixture comprising cefdinir.0.87H₂O and an alcohol in which said cefdinir.0.87H₂O completely dissolves, causing Cefdinir Crystalline Form A to exist in said mixture, and isolating said Cefdinir Crystalline Form A.

Still another embodiment pertains to a process for converting cefdinir.1.14H₂O, to Cefdinir Crystalline Form A by providing a mixture comprising cefdinir.1.14H₂O and an alcohol in which said cefdinir.1.14H₂O completely dissolves, causing Cefdinir Crystalline Form A to exist in said mixture, and isolating said Cefdinir Crystalline Form A.

Still another embodiment pertains to Cefdinir Crystalline Form A prepared by a process comprising providing a mixture comprising cefdinir.1.14H₂O and an alcohol in which said cefdinir.1.14H₂O completely dissolves, causing Cefdinir Crystalline Form A to exist in said mixture, and isolating said Cefdinir Crystalline Form A.

Still another embodiment pertains to a process for converting cefdinir.1.33H₂O, to Cefdinir Crystalline Form A by providing a mixture comprising cefdinir.1.33H₂O and an alcohol in which said cefdinir.1.33H₂O completely dissolves, causing Cefdinir Crystalline Form A to exist in said mixture, and isolating said Cefdinir Crystalline Form A.

Still another embodiment pertains to Cefdinir Crystalline Form A prepared by a process comprising providing a mixture comprising cefdinir.1.33H₂O and an alcohol in which said cefdinir.1.33H₂O completely dissolves, causing Cefdinir Crystalline Form A to exist in said mixture, and isolating said Cefdinir Crystalline Form A.

Still another embodiment pertains to a process for converting cefdinir.1.43H₂O, to Cefdinir Crystalline Form A by providing a mixture comprising cefdinir.1.14H₂O and an alcohol in which said cefdinir.1.43H₂O completely dissolves, causing Cefdinir Crystalline Form A to exist in said mixture, and isolating said Cefdinir Crystalline Form A.

Still another embodiment pertains to Cefdinir Crystalline Form A prepared by a process comprising providing a mixture comprising cefdinir.1.43H₂O and an alcohol in which said cefdinir.1.43H₂O completely dissolves, causing Cefdinir Crystalline Form A to exist in said mixture, and isolating said Cefdinir Crystalline Form A.

Still another embodiment pertains to a process for converting cefdinir trihemihydrate, with or without surface water, to a Cefdinir Crystalline Form A by providing a mixture comprising cefdinir trihemihydrate and an alcohol in which said cefdinir trihemihydrate completely dissolves, causing Cefdinir Crystalline Form A to exist in said mixture, and isolating said Cefdinir Crystalline Form A.

Still another embodiment pertains to Cefdinir Crystalline Form A prepared by a process comprising providing a mixture comprising cefdinir trihemihydrate and an alcohol in which said cefdinir trihemihydrate completely dissolves, causing Cefdinir Crystalline Form A to exist in said mixture, and isolating said Cefdinir Crystalline Form A.

Still another embodiment pertains to a process for converting cefdinir.3.73H₂O, to a Cefdinir Crystalline Form A by providing a mixture comprising cefdinir.3.73H₂O and an alcohol in which said cefdinir.3.73H₂O completely dissolves, causing Cefdinir Crystalline Form A to exist in said mixture, and isolating said Cefdinir Crystalline Form A.

Still another embodiment pertains to Cefdinir Crystalline Form A prepared by a process comprising providing a mixture comprising cefdinir.3.73H₂O and an alcohol in which said cefdinir.3.73H₂O completely dissolves, causing Cefdinir Crystalline Form A to exist in said mixture, and isolating said Cefdinir Crystalline Form A.

Still another embodiment pertains to a process for converting cefdinir.3.76H₂O, to a Cefdinir Crystalline Form A by providing a mixture comprising cefdinir.3.76H₂O and an alcohol in which said cefdinir.3.76H₂O completely dissolves, causing Cefdinir Crystalline Form A to exist in said mixture, and isolating said Cefdinir Crystalline Form A.

Still another embodiment pertains to Cefdinir Crystalline Form A prepared by a process comprising providing a mixture comprising cefdinir.3.76H₂O and an alcohol in which said cefdinir.3.76H₂O completely dissolves, causing Cefdinir Crystalline Form A to exist in said mixture, and isolating said Cefdinir Crystalline Form A.

Still another embodiment pertains to a process for converting cefdinir.3.79H₂O, to a Cefdinir Crystalline Form A by providing a mixture comprising cefdinir.3.79H₂O and an alcohol in which said cefdinir.3.79H₂O completely dissolves, causing Cefdinir Crystalline Form A to exist in said mixture, and isolating said Cefdinir Crystalline Form A.

Still another embodiment pertains to Cefdinir Crystalline Form A prepared by a process comprising providing a mixture comprising cefdinir.3.79H₂O and an alcohol in which said cefdinir.3.79H₂O completely dissolves, causing Cefdinir Crystalline Form A to exist in said mixture, and isolating said Cefdinir Crystalline Form A.

Still another embodiment pertains to a process for converting cefdinir.3.81H₂O, to a Cefdinir Crystalline Form A by providing a mixture comprising cefdinir.3.81H₂O and an alcohol in which said cefdinir.3.81H₂O completely dissolves, causing Cefdinir Crystalline Form A to exist in said mixture, and isolating said Cefdinir Crystalline Form A.

Still another embodiment pertains to Cefdinir Crystalline Form A prepared by a process comprising providing a mixture comprising cefdinir.3.81H₂O and an alcohol in which said cefdinir.3.81H₂O completely dissolves, causing Cefdinir Crystalline Form A to exist in said mixture, and isolating said Cefdinir Crystalline Form A.

Still another embodiment pertains to a process for converting cefdinir anhydrate to Cefdinir Crystalline Form A by providing a mixture comprising cefdinir anhydrate and solvent in which said cefdinir anhydrate completely dissolves, causing Cefdinir Crystalline Form A to exist in said mixture, and isolating said Cefdinir Crystalline Form A.

Still another embodiment pertains to Cefdinir Crystalline Form A prepared by a process comprising providing a mixture comprising cefdinir anhydrate and a solvent in which said cefdinir anhydrate completely dissolves, causing Cefdinir Crystalline Form A to exist in said mixture, and isolating said Cefdinir Crystalline Form A.

Still another embodiment pertains to Cefdinir Crystalline Form A prepared by a process by converting Cefdinir trihemihydrate to Cefdinir Crystalline Form A.

Still another embodiment pertains to Cefdinir Crystalline Form A prepared by a process by converting Cefdinir trihemihydrate to Cefdinir Crystalline Form A, and isolating said Cefdinir Crystalline Form A.

This invention pertains to discovery of a novel crystalline cefdinir anhydrate, novel crystalline iso-structural hydrates of cefdinir lower hydrates, and novel cefdinir trihemihydrate with or without surface water, as well as ways to make them having substantial crystalline, chemical and isomeric purity, ways to characterize them, compositions containing them and methods of treating bacterial infections using them.

Cefdinir is also referred to herein as 7-(2-(2-aminothiazol-4-yl)-2-hydroxyminoacetamido)-3-vinyl-3-cephem-4-carboxylic acid (syn-isomer) and (6R-(6α,7β(Z)))-7-(((2-amino-1,3-thiazol-4-yl)(hydroxyimino)acetyl)amino)-3-ethenyl-8-oxo-5-thia-1-azabicyclo[4.2.0]oct-2-ene-2-carboxylic acid (syn-isomer).

The term “bacterial infections,” means infections resulting from Staphylococcus aureus, Streptococcus pneumoniae, Streptococcus pyogenes, Hemophilus influenzae, Moraxella catarrhalis, E. coli Klebsiella or Proteus mirabilis.

The terms “cefdinir” and “a cefdinir” as used herein, mean amorphous cefdinir, a crystalline cefdinir anhydrate, a crystalline cefdinir lower hydrate and iso-structural hydrates thereof, crystalline cefdinir trihemihydrate with or without surface water, microcrystalline cefdinir, cefdinir in solution, semisolid cefdinir and mixtures thereof.

The terms “crystalline” and “microcrystalline,” as used herein, mean having a regularly repeating arrangement of molecules which is maintained over a long range or external face planes.

The term “crystalline cefdinir,” as used herein, means a particular crystalline cefdinir, including a crystalline cefdinir of this invention.

The term “crystalline cefdinir of this invention,” as used herein, means crystalline cefdinir trihemihydrate with or without surface water, a crystalline iso-structural hydrate of a cefdinir lower hydrate, and a crystalline cefdinir anhydrate characterized, when measured with radiation at 1.54178 Å, comprising a powder diffraction pattern having 2θ values of about 5.5°, 10.9°, 12.6°, 14.7°, 16.6°, 21.8° and 27.3°.

The term “amorphous,” as used herein, means a supercooled liquid substance or a viscous liquid which appears as a solid but does not have a regularly repeating arrangemant of molecules which is maintained over a long range. Amorphous substances do not have a melting point but soften or flow above a certain temperature known as the glass transition temperature.

The term “semisolid cefdinir,” as used herein, means a combination of cefdinir and solvent in a gelatinous enough state to prevent its passage through a semi-permeable membrane or filter.

The term “crystalline cefdinir anhydrate,” as used herein, means crystalline cefdinir with a water content of 0% in the crystal.

The term “hydrate,” as used herein, means having water associated therewith.

The term “crystalline cefdinir lower hydrate,” as used herein, means crystalline cefdinir.0.5H₂O or crystalline cefdinir.1.5H₂O, each of which is characterized in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.9° (0,0,1), 8.1° (2,0,−1), 11.8° (2,0,−2), 15.7° (4,0,0), 16.2° (2,0,2), 22.6° (2,0,−4) and 21.0° (3,1,1), or by a combination thereof, wherein each peak is shown with its accompanying Miller Index (h,k,l) value.

The term “iso-structural hydrate of a cefdinir lower hydrate,” as used herein, means a crystalline hydrate of cefdinir.0.5H₂O or cefdinir.1.5H₂O characterized by substantially similar unit cell parameters and powder X-ray diffraction pattern of the corresponding lower hydrate but with a different water content in the unit cell, wherein the term “substantially similar,” as used herein, means falling within the range of the unit cell parameters.

Examples of iso-structural hydrates of cefdinir lower hydrates of this invention include, but are not limited to, cefdinir.0.87H₂O (3.8), cefdinir.1.14H₂O (4.9), cefdinir. 1.33H₂O cefdinir.1.43H₂O (6.1) and the like, wherein the corresponding water content (percent of water per sample) is shown in parenthesis following each example.

The term “crystalline cefdinir trihemihydrate,” as used herein, means cefdinir.3.5H₂O characterized in the monoclinic crystal system and C2 space group, when measured with radiation at 0.7107 Å, by respective lattice parameters a, b and c of 23.95 Å±0.03 Å, 4.984 Å±0.006 Å and 15.87 Å±0.01 Å and β of 108.88°±0.02° or, when measured with radiation at 1.54178 Å, comprising a powder diffraction pattern having 2θ values of about 5.4° (0,0,1), 10.7° (0,0,2), 14.2° (2,0,2), 15.2° (4,0,0), 21.4° (0,0,4), 29.2° (2,0,5) and 30.6° (8,0,0), or by a combination thereof, wherein each peak is shown with its accompanying Miller Index (h,k,l) value.

It is meant to be understood that when peak positions are further used to identify a particular crystalline form of a compound when unit cell parameters of the compound are used in combination therewith, any one peak position or combination of peak positions may be used to further identify the particular crystalline form.

A sample of crystalline cefdinir trihemihydrate may or may not have associated therewith surface water. Examples of crystalline cefdinir trihemihydrate having surface water associated therewith include, but are not limited to cefdinir.3.67H₂O (14.33), cefdinir.3.73H₂O (14.53), cefdinir.3.76H₂O (14.63), cefdinir.3.77H₂O (14.68), cefdinir.3.79H₂O (14.73), cefdinir.3.81H₂O (14.80) and the like, wherein the corresponding percentage of water per sample is shown in parenthesis following each example.

Unless stated otherwise, percentages herein are weight/weight (w/w) percentages.

Without being limited by theory, the unit cell of crystalline cefdinir.1.5H₂O has three water molecules per two cefdinir molecules and tunnels in which iso-structural water may reside. One of the water molecules is more labile than the others and an oxygen atom of one of the water molecules is shared between two unit cells. A continuum of the amount of iso-structural water exists for cefdinir lower hydrates and is dependent on the amount of water available to it through atmospheric humidity.

The unit cell of crystalline cefdinir.3.5H₂O has seven water molecules per two cefdinir molecules and, because the tunnels into which iso-structural water may reside are full, is able to accommodate only about 0.1% to about 3% surface water.

The term “substantial crystalline purity,” as used herein, means at least about 95% crystalline purity, preferably about 97% crystalline purity, more preferably about 99% crystalline purity, and most preferably about 100% crystalline purity.

The term “crystalline purity,” as used herein, means percentage of a particular crystalline form of a compound in a sample which may contain the amorphous form of the compound, one or more than one other crystalline forms of the compound other than the particular crystalline form of the compound, or a mixture thereof.

The term “substantial chemical purity,” as used herein, means about 95% chemical purity, preferably about 97% chemical purity, more preferably about 98% chemical purity, and most preferably about 100% chemical purity.

The term “chemical purity,” as used herein, means percentage of a particular compound in a sample. For example, crystalline cefdinir, may contain varying amounts of impurities including, but not limited to, (2Z)-N-((5aR,6R)-3-methyl-1,7-dioxo-1,4,6,7-tetrahydro-3H,5aH-azeto[2,1-b]furo[3,4-d][1,3]thiazin-6-yl)-2-(2-amino-1,3-thiazol-4-yl)-2-(hydroxyimino)ethanamide, (2R)-(((2Z)-2-(2-amino-1,3-thiazol-4-yl)-2-(hydroxyimino)acetyl)amino)((2R)-5-methyl-7-oxo-1,2,5,7-tetrahydro-4H-furo[3,4-d][1,3]thiazin-2-yl)ethanoic acid, (2Z)-2-(2-amino-1,3-thiazol-4-yl)-2-(hydroxyimino)-N-(((2R)-5-methyl-7-oxo-1,2,5,7-tetrahydro-4H-furo[3,4-d][1,3]thiazin-2-yl)methyl)ethanamide, (2Z)-2-(2-amino-1,3-thiazol-4-yl)-N-(2,2-dihydroxyethyl)-2-(hydroxyimino)ethanamide, (2R,5Z)-2-[(R)-(((2Z)-2-(2-amino-1,3 -thiazol-4-yl)-2-(hydroxyimino)ethanoacetyl)(carboxy)methyl)-5-ethylidene-5,6-dihydro-2H-1,3 -thiazine-4-carboxylic acid, (2R,5Z)-2-((((2Z)-2-(2-amino-1,3-thiazol-4-yl)-2-(hydroxyimino)acetyl)amino)methyl)-5-ethylidene-5,6-dihydro-2H-1,3-thiazine-4-carboxylic acid, (((2Z)-2-(2-amino-1,3-thiazol-4-yl)-2-(hydroxyimino)acetyl)amino)acetic acid, (6R,7R)-7-(((2Z)-2-(2-amino-1,3-thiazol-4-yl)-2-(hydroxyimino)acetyl)amino)-3-methyl-8-oxo-5-thia-1-azabicyclo[4.2.0]oct-2-ene-2-carboxylic acid, (6R,7R)-7-(((2-amino-1,3-thiazol-4-yl)acetyl)amino)-8-oxo-3-vinyl-5-thia-1-azabicyclo[4.2.0]oct-2-ene-2-carboxylic acid, (6R,7R)-7-(((4-hydroxyisoxazol-3-yl)carbonyl)amino)-8-oxo-3-vinyl-5-thia-1-azabicyclo[4.2.0]oct-2-ene-2-carboxylic acid, (6R,7R)-7-(((2Z)-2-(2-amino-1,3-thiazol-4-yl)-2-(hydroxyimino)acetyl)amino)-8-oxo-3-vinyl-5-thia-1-azabicyclo[4.2.0]oct-2-ene-2-carboxylic acid 5-oxide, (6R,7R)-7-(((2-amino-1,3 -thiazol-4-yl)(oxo)acetyl)amino)-8-oxo-3-vinyl-5-thia-1-azabicyclo[4.2.0]oct-2-ene-2-carboxylic acid, (2E)-2-(2-amino-1,3-thiazol-4-yl)-2-(hydroxyimino)-N-(((2R)-5-methyl-7-oxo-1,2,5,7-tetrahydro-4H-furo[3,4-d][1,3]thiazin-2-yl)methyl)ethanamide, mixtures thereof and the like.

Cefdinir may exist as isomers wherein the oxime moiety thereof is in the syn-configuration, the anti-configuration, or a mixture of syn- and anti- configurations.

The term “substantial isomeric purity,” as used herein, means having an isomeric excess greater than about 95%, preferably greater than about 97%, more preferably greater than about 99%, and most preferably about 100%.

The term “isomeric excess,” as used herein, means amount of one isomer of a compound in a sample which may contain another isomer of the same compound.

For example, crystalline cefdinir, may contain varying amounts of (6R-(6α,7β(E)))-7-(((2-amino-1,3-thiazol-4-yl)(hydroxyimino)acetyl)amino)-3-ethenyl-8-oxo-5-thia-1-azabicyclo[4.2.0]oct-2-ene-2-carboxylic acid (cefdinir, anti isomer).

This invention also includes mixtures comprising a crystalline cefdinir of this invention in combination with one or more than one other crystalline cefdinirs of this invention and also includes mixtures comprising one or more than one crystalline cefdinirs of this invention in combination with one or more than one cefdinirs including, but not limited to, amorphous cefdinir, microcrystalline Cefdinir, Cefdinir Crystalline Form A, a crystalline cefdinir having a water content of 4.11%, when measured with radiation at 1.54178 Å, comprising a powder diffraction pattern having 2θ values of about 11.7°, 16.1°, 18.6°, 21.2°, 22.3°, 23.6°, 24.4°, 26.2° and 28.0°, a crystalline cefdinir having a water content of 1.05%, when measured with radiation at 1.54178 Å comprising a powder diffraction pattern having 2θ values of about 11.8°, 16.2°, 18.6°, 19.4°, 21.0°, 21.2°, 22.3°, 24.5°, 25.7° and 36.3°, a crystalline cefdinir anhydrate when measured with radiation at 1.54178 Å comprising a powder diffraction pattern having 2θ values of about 11.4°, 16.1°, 18.4°, 19.8°, 21.9°, 22.2°, 23.5°, 24.7°, 27.9°0 and 31.6°, a crystalline cefdinir having a water content of 5.5% to 7.0%, when measured with radiation at 1.54178 Å, comprising a powder diffraction pattern having 2θ values of 5.8°±0.2°, 7.7°±0.2°, 8.0°±0.2°, 11.7°±0.2°, 15.6°±0.2°, 16.1°±0.2°, 18.6°±0.2°, 20.9°±0.2°, 22.2°±0.2°, 24.4°±0.2° and 25.6°±0.2°, and mixtures thereof.

It is meant to be understood that each component of mixtures of two or more crystalline cefdinirs may have varying degrees of chemical and isomeric purity and that, in a preferred embodiment for the practice of this invention, in mixtures comprising different forms of cefdinir, each cefdinir in the mixture is substantially chemically and isomerically pure.

The term “solvent,” as used herein, means a liquid in which a compound is soluble or partially soluble enough at a given concentration to dissolve or partially dissolve the compound.

The term “anti-solvent,” as used herein, means a liquid in which a compound is insoluble enough at a given concentration to be effective for precipitating that compound from a solution.

Solvents and anti-solvents may be mixed with or without separation of phases.

It is meant to be understood that, because many solvents and anti-solvents contain impurities, the level of impurities in solvents and anti-solvents for the practice of this invention, if present, are at a low enough concentration that they do not interfere with the intended use of the solvent in which they are present.

The term “acid,” as used herein, means a compound having at least one acidic proton. Examples of acids for the practice of this invention include, but are not limited to, hydrochloric acid, hydrobromic acid, trifluoroacetic acid, trichloroacetic acid, sulfuric acid, phosphoric acid and the like.

The term “base,” as used herein, means a compound capable of accepting a proton. Examples of bases for the practice of this invention include, but are not limited to, sodium carbonate, sodium bicarbonate, potassium carbonate, potassium bicarbonate triethylamine, diisopropylethylamine and the like.

The term “solvent,” as used herein, means a liquid which is capable of dissolving a cefdinir. Examples of solvents for the practice of this invention include, but are not limited to, water and organic solvents including, but not limited to, methanol, ethanol, tetrahydrofuran, acetonitrile, N,N-dimethylformamide, dimethylsulfoxide, ethyl acetate, isopropyl acetate, pyridine and the like.

Causing Cefdinir Crystalline Form A to exist in a mixture comprising cefdinir and solvent, wherein the cefdinir has completely dissolved, is known as nucleation.

For the practice of this invention, nucleation may be made to occur by means such as solvent removal, temperature change, solvent-miscible anti-solvent addition, solvent-immiscible anti-solvent addition, seed crystal addition of Cefdinir Crystalline Form A, chafing or scratching the interior of the container, preferably a glass container, in which nucleation is meant to occur with an implement such as a glass rod or a glass bead or beads, or a combination of the foregoing.

For the practice of this invention, nucleation may be followed by crystal growth, accompanied by crystal growth, or followed and accompanied by crystal growth during which, and as a result of which, the percentage of Cefdinir Crystalline Form A increases.

It is meant to be understood that airborne seed crystals of crystalline Cefdinir Crystalline Form A may also cause nucleation in a mixture of Cefdinir Crystalline Form A and solvent in which the Cefdinir Crystalline Form A has completely dissolved.

The term “seed crystal,” as used herein, means a particular crystalline form of a substance having mass. It is meant to be understood that such a crystal may be small enough to be airborne or invisible to the eye without means of detection.

The term “isolating” as used herein, means separating a crystalline cefdinir and solvent, anti-solvent, or a mixture of solvent anti-solvent. This is typically accomplished by means such as centrifugation, filtration with or without vacuum, filtration with positive pressure, distillation, evaporation or a combination thereof.

A therapeutically acceptable amount of a cefdinir depend on recipient of treatment, disorder being treated and severity thereof, composition containing it, time of administration, route of administration, duration of treatment, its potency, its rate of clearance and whether or not another drug is co-administered. The amount of a cefdinir used to make a composition to be administered daily to a patient in a single dose or in divided doses is from about 0.03 to about 200 mg/kg body weight. Single dose compositions contain these amounts or a combination of submultiples thereof.

A cefdinir may be administered with or without an excipient and with or without amorphous cefdinir. Excipients include but are not limited to, for example, encapsulating materials and additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, mixtures thereof and the like.

Excipients for preparation of compositions comprising and made with a cefdinir to be administered orally in solid dosage form include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, mixtures thereof and the like. Excipients for preparation of compositions comprising and made with a cefdinir to be administered ophthalmically or orally in liquid dosage forms include, for example, 1,3-butylene glycol, castor oil, corn oil, cottonseed oil, ethanol, fatty acid esters of sorbitan, germ oil, groundnut oil, glycerol isopropanol, olive oil, polyethylene glycols, propylene glycol, sesame oil, water, mixtures thereof and the like. Excipients for preparation of compositions comprising and made with a cefdinir to be administered osmotically include, for example, chlorofluorohydrocarbons, ethanol, water, mixtures thereof and the like. Excipients for preparation of compositions comprising and made with a cefdinir to be administered parenterally include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water, mixtures thereof and the like. Excipients for preparation of compositions comprising and made with a cefdinir to be administered rectally or vaginally include, but are not limited to, cocoa butter, polyethylene glycol, wax, mixtures thereof and the like.

Amorphous cefdinir may be prepared as described in U.S. Pat. No. 4,559,334, of which column 2, line 15 to column 11 line 7 and column 12, line 20 to column 20, line 5 are hereby incorporated by reference into this specification.

Crystalline cefdinir may be prepared as described in U.S. Pat. No. 4,935,507, of which column 4, line 36 to column 12, line 45 and column 13, lines 6-64 and column 14, line 20 to column 16, line 7 are hereby incorporated by reference into this specification. For use herein, crystalline cefdinir Form A was obtained from Fujisawa Corporation (Osaka, Japan).

The following examples are presented to provide what is believed to be the most useful and readily understood description of procedures and conceptual aspects of this invention.

### EXAMPLE 1

### benzhydryl(6R-(6α,7β(Z)))-7-(((2-amino-1,3-thiazol-4-yl)-2-(hydroxyimino)acetyl)amino)-8-oxo-3-vinyl-5-thia-1-azabicyclo[4.2.0]oct-2-ene-2-carboxylate

A mixture of benzhydryl (6R,7R)-7-((4-bromo-3-oxobutanoyl)amino)-8-oxo-3-vinyl-5-thia-1-azabicyclo[4.2.0]oct-2-ene-2-carboxylate (10 g) in dichloromethane (70 mL) and acetic acid (25 mL) at −3-5° C. was treated with isoamylnitrite (3.5 mL), stirred for 40 minutes, treated with acetylacetone (4 g), stirred for 30 minutes at 5° C., treated with thiourea (3 g), stirred for 3 hours, treated with ethyl acetate (70 mL) and diisopropyl ether (100 mL), and filtered. The filtrant was dried under vacuum.

### EXAMPLE 2

### (6R-(6α,7β(Z)))-7-(((2-amino-1,3-thiazol-4-yl)(hydroxyimino)acetyl)amino)-3-ethenyl-8-oxo-5-thia-1-azabicyclo[4.2.0]oct-2-ene-2-carboxylic acid (cefdinir, syn-isomer)

A mixture of 2,2,2-trifluroacetic acid and anisole at 5° C. to 7° C. was treated with EXAMPLE 1, stirred for 1 hour at 5° C., added to diisopropyl ether (150 mL), and filtered. The filtrant was dissolved in THF (10 mnL) and ethyl acetate (10 mL), and the mixture was extracted with aqueous sodium bicarbonate. The extract was washed with ethyl acetate while keeping the pH at 5, adjusted to pH 2.2 with 10% hydrochloric acid, stirred for 1 hour at 0° C., and filtered. The filtrant was dried under vacuum.

Alternatively, boron trifluoride etherate (5 mL) at 10° C. was treated with EXAMPLE 1 (5 g) in anisole (20 mL) and acetic acid (5 mL), stirred for 20 minutes, poured into 1:1:1 THF/ethyl acetate/water (300 mL), and adjusted to pH 6 with 20% aqueous sodium hydroxide. The water layer was isolated, adjusted to pH 6 if necessary, washed with ethyl acetate, and eluted through aluminum oxide with 3% aqueous sodium acetate. Elutes containing desired product were collected, adjusted to pH 4 with 10% hydrochloric acid, and eluted through nonionic absorption resin Diaion HP-20® (Mitsubishi Chemical Industries) with 20% aqueous acetone. Elutes containing desired product were collected, concentrated, adjusted to pH 2 with 10% hydrochloric acid, and filtered.

### EXAMPLE 3

### crystalline(6R-(6α,7β(Z)))-7-(((2-amino-1,3-thiazol-4-yl)(hydroxyimino)acetyl)amino)-3-ethenyl-8-oxo-5-thia-1-azabicyclo[4.2.0]oct-2-ene-2-carboxylic acid.3.5H₂O (crystalline cefdinir trihemihydrate)

A mixture of EXAMPLE 2 (805 mg) in 1:1 ethanol:ethyl acetate was treated with 6 drops of concentrated H₂SO₄ with intermittent sonication. The mixture cleared, then a yellow semisolid formed. This mixture was transferred to a separatory funnel and treated with water to provide a white suspension. This mixture was vortex-mixed, centrifuged to provide sediment and a liquid layer, and decanted, and this procedure was repeated until a liquid layer pH of about 3.5 was attained.

In another experiment, after the yellow semisolid formed, the mixture was treated with water to provide the white suspension and transferred to each of six centrifuge tubes containing water (9 mL) to provide a total volume of 12 mnL in each tube. The suspension in each was centrifuged, and the sediment and liquid layer of each were separated. This procedure was repeated until a liquid layer pH of about 3.5 for each was attained.

### EXAMPLE 4

### crystalline(6R-(6α,7β(Z)))-7-(((2-amino-1,3-thiazol-4-yl)(hydroxyimino)acetyl)amino)-3-ethenyl-8-oxo-5-thia-1-azabicyclo[4.2.0]oct-2-ene-2-carboxylic acid.1.5H₂O (crystalline cefdinir sesquihydrate)

Crystalline cefdinir trihemihydrate, with or without surface water, was heated at 75° C. for 30 minutes.

### EXAMPLE 5

### crystalline(6R-(6α,7β(Z)))-7-(((2-amino-1,3-thiazol-4-yl)(hydroxyimino)acetyl)amino)-3-ethenyl-8-oxo-5-thia-1-azabicyclo[4.2.0]oct-2-ene-2-carboxylic acid.1.5H₂O (crystalline cefdinir sesquihydrate)

Crystalline cefdinir sesquihydrate or an iso-structural hydrate thereof was heated at 150° C. for 30 minutes. Vacuum drying crystalline cefdinir trihemihydrate or sesquihydrate or an iso-structural hydrate thereof, will also produce crystalline cefdinir anhydrate.

### EXAMPLE 6

### Cefdinir Crystalline (syn-isomer) Form A

Cefdinir Crystalline Form A may be prepared as described in U.S. Pat. No. 4,935,507, or which column 13, lines 42-64 are hereby incorporated by reference into this specification.

Generally, suitable examples of solutions containing cefdinir include, for example, an aqueous solution of an alkali metal salt of cefdinir. The solution containing cefdinir is acidified, if necessary, after elution through a column of activated charcoal, nonionic adsorption resin, alumina or acidic aluminum oxide. Acidification may be achieved by adding an acid such as hydrochloric acid or the like preferably between about 25° C. to about 40° C., more preferably, from 15° to 40° C. The amount of the acid to be added should make the pH of the solution about 1 to about 4.

Specifically, cefdinir (syn-isomer) (29.55 g) in water (300 mnL) is adjusted to pH 6.0 with saturated sodium bicarbonate solution, and column chromatographed on activated charcoal with 20% aqueous acetone. Elutes containing product are combined and concentrated to 500 mL, warmed to 35° C., adjusted to pH 1.8 with 4M hydrochloric acid, and filtered. Alternatively cefdinir (syn-isomer) (0.5 g) in methanol (10 mL) at 35° C. is treated with water (1.5 mL) at 35° C., stirred for 3 minutes, cooled to room temperature, and filtered.

Alternatively, cefdinir is dissolved in methanol, stirred under warming, preferably below about 40° C., treated with water which is at about the same temperature as the solution, cooled and filtered.

It is meant to be understood that amorphous cefdinir, a crystalline cefdinir anhydrate, a crystalline cefdinir lower hydrate or an iso-structural hydrates thereof, crystalline cefdinir trihemihydrate with or without surface water, microcrystalline cefdinir and mixtures thereof may also be used to prepare Cefdinir Crystalline Form A.

During the crystallization of Cefdinir Crystalline form A, it is preferable to keep the solution slightly beyond the saturation point. Cefdinir thus obtained by this process can be collected by filtration and dried conventionally.

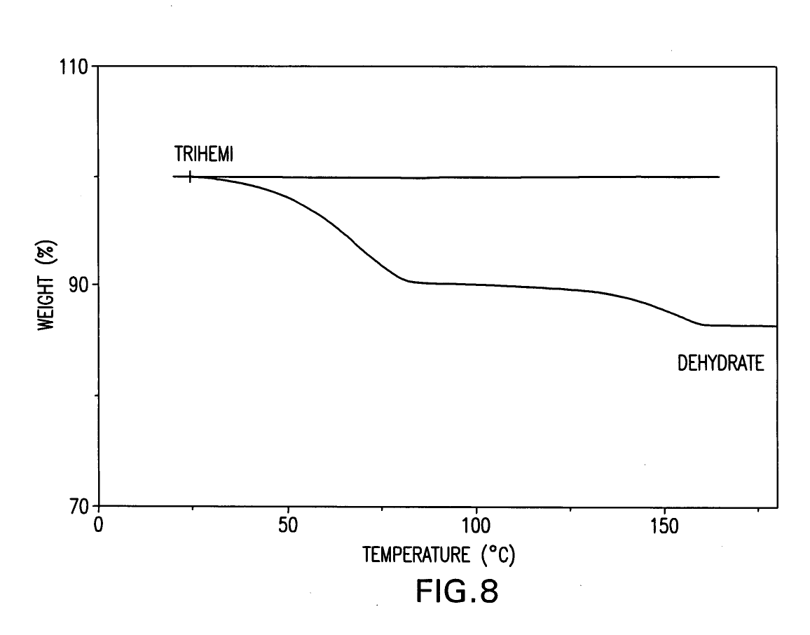

Thermogravimetric analyses (TGA) were performed in TA Instruments TG2950 (TA Instruments, New Castle, Del.). Samples were scanned at 10° C./minute with a dry nitrogen purge of 60 mL/minute. Results of the TGA (FIG. 8) show the transformation of cefdinir trihemihydrate to cefdinir sesquihydrate to the cefdinir anhydrate of this invention.

Powder X-ray diffraction was performed using an XDS-2000/X-ray diffractometer equipped with a 2 kW normal focus X-ray tube and a Peltier cooled germanium solid-state detector (Scintag Inc., Sunnyvale, Calif.). The data were processed using DMSNT software (version 1.37). The X-ray source was a copper filament (Cu—Kα at 1.54178 Å) operated at 45 kV and 40 mA. The alignment of the goniometer was checked daily using a Corundum standard. The sample was placed in a thin layer (with no prior grinding) onto a zero background plate and continuously scanned at a rate of 2° 2θ per minute over a range of 2°-40° 2θ.

It is meant to be understood that relative intensities of peak heights in a PXRD pattern may vary and will be dependent on variables such as the temperature, size of crystal size or morphology, sample preparation, or sample height in the analysis well of the X-ray diffractometer.

It is also meant to be understood that peak positions may vary when measured with different radiation sources. For example, Cu—Kα₁, Mo—Kα, Co—Kα and Fe—Kα radiation, having wavelengths of 1.54060 Å, 0.7107 Å, 1.7902 Å and 1.9373 Å, respectively, may provide peak positions which differ from those measured with Cu—Kα radiation, which has a wavelength of 1.5478 Å.

The term “about” preceding a series of peak positions is meant to include all of the peak positions of the group which it precedes.

The term “about” preceding a series of peak positions means that all of the peaks of the group which it precedes are reported in terms of angular positions (two theta) with an allowable variability of ±0.1° as specified by the U.S. Pharmacopeia, pages 1843-1884 (1995). The variability of ±0.1° is intended to be used when comparing two powder X-ray diffraction patterns. In practice, if a diffraction pattern peak from one pattern is assigned a range of angular positions (two theta) which is the measured peak position ±0.1° and if those ranges of peak positions overlap, then the two peaks are considered to have the same angular position. For example, if a peak from one pattern is determined to have a position of 5.2°, for comparison purposes the allowable variability allows the peak to be assigned a position in the range of 5.1°-5.3°. If a peak from another diffraction pattern has a peak position of 5.3°, for comparison purposes, the allowable variability allows the peak to be assigned a position in the range of 5.2°-5.4°. Because there is overlap between the two ranges of peak positions (i.e., 5.1°-5.3° and 5.2°-5.4°) the two peaks being compared are considered to have the same angular position.

Accordingly, for example, the phrase “about 5.9°, 8.1°, 11.8°, 15.7°, 16.2°, 22.6° and 23.1°,” as used herein, means about 5.9°, about 8.1°, about 11.8°, about 15.7°, about 16.2°, about 22.6° and about 23.1° and also means 5.9°±0.1°, 8.1°±0.1°, 11.8°±0.1°, 15.7°±0.1°, 16.2°±0.1°, 22.6°±0.1° and 23.1°±0.1°.

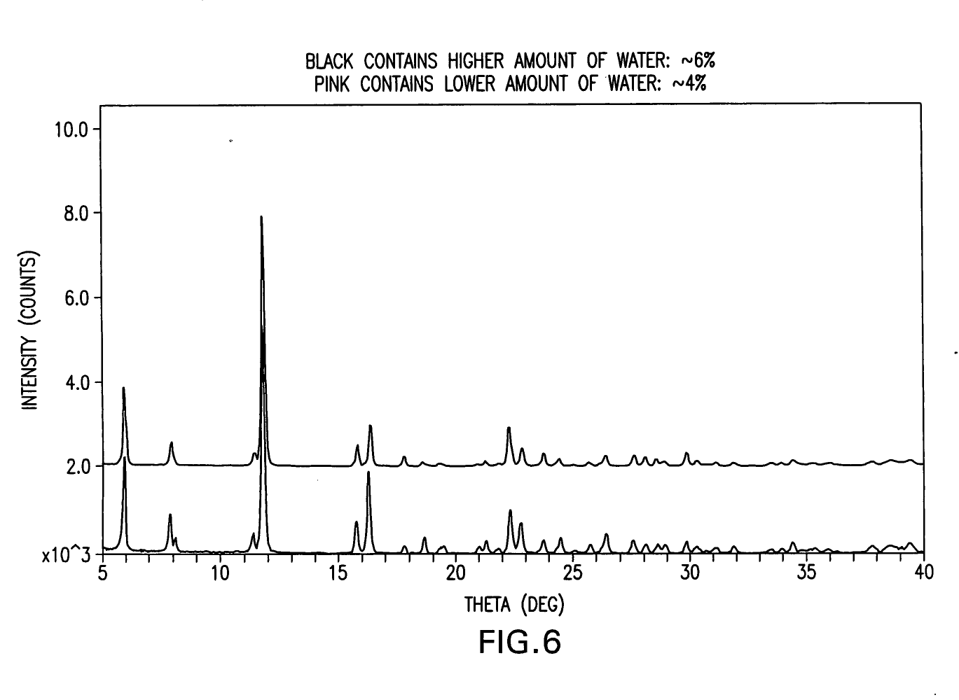

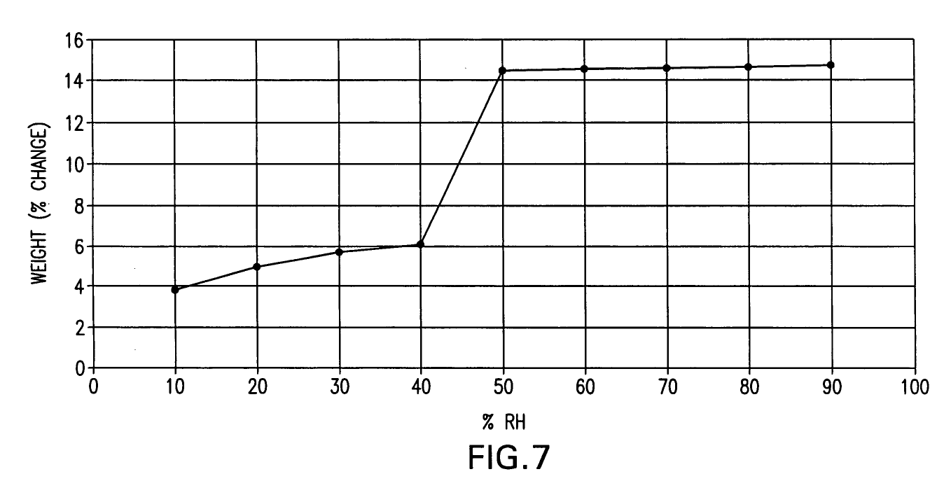

Dynamic Moisture Sorption/Desorption Gravimetric (DMSG) analysis was performed for the cefdinir lower hydrates. A vacuum moisture balance (MB 300G, VTI Corporation) was used to study the moisture sorption and desoprtion. Samples were dried at 50° C. under vacuum to constant weight. Relative humidity was increased to 90% in 10% increments. If the sample weight remained unchanged (i.e. changed by less than about 3 mg/15 min), the moisture content was recorded. The balance was calibrated before the experiment and the accuracy of the relative humidity measurement was verified with polyvinylpyrrolidone K90. FIG. 7 shows the moisture desorption isotherm of the hydrates of the present invention. Sharp steps, for example with relative humidity changes from 40% to 50%, occur when the crystal undergoes a phase change (i.e. a crystalline structure change). Relatively flat regions represent a unique phase (i.e. where the crystalline structure does not change and is more physically stable). Increases in the relative humidity from 10% to almost 40%, results in the formation of crystalline iso-structural hydrates. The cefdinir iso-structural hydrates varied but maintained the same crystalline structure and PXRD patterns (FIG. 6). An increase in the relative humidity from 40% to 50% caused a crystalline structure change, and further increase of relative humidity from 50% to 90% caused cefdinir trihemihydrate (having a water content of about 14%) to form.

TABLE 1 summarizes the typical weight changes of cefdinir relative to changes in relative humidity. The weight changes are expressed by percentage of water content and by the calculated molar content of water.

TABLE 1

| | | Calc'd Moles of |
|---|---|---|
| % Relative Humidity | Percent Water | Water |
| 10.24 | 3.80 | 0.87 |
| 20.24 | 4.94 | 1.14 |
| 30.17 | 5.71 | 1.33 |
| 40.19 | 6.13 | 1.43 |
| 50.08 | 14.53 | 3.73 |
| 60.10 | 14.63 | 3.76 |
| 70.00 | 14.68 | 3.77 |
| 79.94 | 14.73 | 3.79 |
| 80.07 | 14.33 | 3.67 |
| 89.90 | 14.80 | 3.81 |

The foregoing is meant to be illustrative of the invention and not intended to limit it to the disclosed embodiments. Variations and changes obvious to one skilled in the art are intended to be within the scope and nature of the invention as defined in the claims.

We claim:

1. Isolated crystalline cefdinir trihemihydrate, with or without surface water, said isolated crystalline cefdinir trihemihydrate characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

2. Isolated crystalline cefdinir trihemihydrate, with or without surface water, said isolated crystalline cefdinir trihemihydrate having substantial crystalline purity, said isolated crystalline cefdinir higher hydrate characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

3. Isolated crystalline cefdinir trihemihydrate, with or without surface water, having substantial crystalline purity and substantial chemical purity, said isolated crystalline cefdinir trihemihydrate characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

4. Isolated crystalline cefdinir trihemihydrate, with or without surface water, having substantial crystalline purity, substantial chemical purity and substantial isomeric purity, said isolated cefdinir trihemihydrate characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

5. A pharmaceutical composition comprising isolated crystalline cefdinir trihemihydrate, with or without surface water, said isolated crystalline cefdinir trihemihydrate characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

6. A method of treating bacterial infection in a human comprising administering thereto a therapeutically effective amount of isolated crystalline cefdinir trihemihydrate, with or without surface water, said isolated crystalline cefdinir trihemihydrate characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

7. A mixtures comprising Cefdinir Crystalline Form A and a crystalline cefdinir trihemihydrate, with or without surface water, said crystalline cefdinir trihemihydrate characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

8. A pharmaceutical composition comprising Cefdinir Crystalline Form A and a crystalline cefdinir trihemihydrate, with or without surface water, said crystalline cefdinir trihemihydrate characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

9. A method of treating bacterial infection in a human comprising administering thereto a therapeutically effective amount of a mixture of Cefdinir Crystalline Form A and an crystalline cefdinir trihemihydrate, with or without surface water, said crystalline cefdinir trihemihydrate characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02°or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

10. A mixture comprising Cefdinir Crystalline Form A and crystalline cefdinir.3.73H₂O, said crystalline cefdinir.3.73H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

11. A pharmaceutical composition comprising Cefdinir Crystalline Form A and crystalline cefdinir.3.73H₂O, said crystalline cefdinir.3.73H₂O characterized, in the monoclinic crystal system and C2 space group when measured with radiation at 0.7107 Å, by respective lattice parameters a, band c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

12. A method of treating bacterial infection in a human comprising administering thereto a therapeutically effective amount of a mixture of Cefdinir Crystalline Form A and crystalline cefdinir.3.73H₂O, said crystalline cefdinir.3.73H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

13. A mixture comprising Cefdinir Crystalline Form A and crystalline cefdinir.3.76H₂O, said crystalline cefdinir.3.76H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

14. A pharmaceutical composition comprising Cefdinir Crystalline Form A and crystalline cefdinir.3.76H₂O, said crystalline cefdinir.3.76H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

15. A method of treating bacterial infection in a human comprising administering thereto a therapeutically effective amount of a mixture of Cefdinir Crystalline Form A and crystalline cefdinir.3.76H₂O, said crystalline cefdinir.3.76H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

16. A mixture comprising Cefdinir Crystalline Form A and crystalline cefdinir.3.79H₂O, said crystalline cefdinir.3.79H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

17. A pharmaceutical composition comprising Cefdinir Crystalline Form A and crystalline cefdinir.3.79H₂O, said crystalline cefdinir.3.79H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

18. A method of treating bacterial infection in a human comprising administering thereto a therapeutically effective amount of a mixture of Cefdinir Crystalline Form A and crystalline cefdinir.3.79H₂O, said crystalline cefdinir.3.79H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

19. A mixture comprising Cefdinir Crystalline Form A and crystalline cefdinir.3.81H₂O, said crystalline cefdinir.3.81H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

20. A pharmaceutical composition comprising Cefdinir Crystalline Form A and crystalline cefdinir.3.81H₂O, said crystalline cefdinir.3.81H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

21. A method of treating bacterial infection in a human comprising administering thereto a therapeutically effective amount of a mixture of Cefdinir Crystalline Form A and crystalline cefdinir.3.81H₂O, said crystalline cefdinir.3.81H₂O characterized, in the monoclinic crystal system and C2 space group when measured with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof.

22. A process for making crystalline cefdinir trihemihydrate, with or without surface water, said process comprising:
providing a mixture comprising cefdinir and solvent;
causing crystalline cefdinir trihemihydrate to exist in said mixture, said crystalline cefdinir trihemihydrate, when isolated with or without surface water, and characterized in the monoclinic crystal system and C2 space group with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof, and
isolating said crystalline cefdinir trihemihydrate.

23. Crystalline cefdinir trihemihydrate, with or without surface water, prepared by a process comprising providing a mixture comprising cefdinir and solvent, causing crystalline cefdinir trihemihydrate to exist in said mixture, said crystalline cefdinir trihemihydrate, when isolated with or without surface water and characterized in the monoclinic crystal system and C2 space group with radiation at 0.7107 Å, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof, and isolating said crystalline cefdinir trihemihydrate.

24. A process for making crystalline cefdinir trihemihydrate, with or without surface water, said process comprising:
providing a mixture comprising semisolid cefdinir, ethyl acetate, ethanol and acid or base;
adding water to said mixture to form crystalline cefdinir trihemihydrate, said crystalline cefdinir trihemihydrate, when isolated with or without surface water and characterized with radiation at 0.7107 Å in the monoclinic crystal system and C2 space group, by respective lattice parameters a, band c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof; and
isolating said crystalline cefdinir trihemihydrate.

25. Crystalline cefdinir trihemihydrate, with or without surface water, prepared by a process comprising providing a mixture comprising semisolid cefdinir, ethyl acetate, ethanol and acid or base, adding water to said mixture to form crystalline cefdinir trihemihydrate, said crystalline cefdinir trihemihydrate, when isolated with or without surface water and characterized with radiation at 0.7107 Å in the monoclinic crystal system and C2 space group, by respective lattice parameters a, b and c of 23.77 Å±0.02 Å, 5.007 Å±0.004 Å and 16.76 Å±0.01 Å and β of 100.29°±0.02° or, when measured with radiation at 1.54178 Å, comprising a powder diffraction pattern having 2θ values of about 5.3°, 10.6°, 14.1°, 15.1°, 21.3°, 29.1° and 30.5°, or by a combination thereof; and isolating said crystalline cefdinir trihemihydrate.

26. A process for making crystalline cefdinir triherihydrate with or without surface water comprising converting 7-amino-3-vinyl-3-cephem-4-carboxylic acid ((6R,7R)-7-amino-8-oxo-3-vinyl-5-thia-1-azabicyclo[4.2.0]oct-2-ene-2-carboxylic acid) to crystalline cefdinir trihemihydrate, with or without carboxylic acid protection and deprotection steps, said process further comprising converting semisolid cefdinir containing at least one residual solvent from said process to crystalline cefdinir trihemihydrate.

27. Crystalline cefdinir trihemihydrate prepared by a process comprising converting 7-amino-3-vinyl-3-cephem-4-carboxylic acid ((6R,7R)-7-amino-8-oxo-3-vinyl-5-thia-1-azabicyclo[4.2.0]oct-2-ene-2-carboxylic acid) to crystalline cefdinir trihemihydrate, with or without carboxylic acid protection and deprotection steps, and further comprising converting semisolid cefdinir containing at least one residual solvent from said process to crystalline cefdinir trihemihydrate.

28. A process for making crystalline cefdinir trihemihydrate comprising making a mixture of semisolid cefdinir and solvent and converting said semisolid cefdinir to crystalline cefdinir trihemihydrate.

29. Crystalline cefdinir trihernihydrate prepared by a process comprising making a mixture of semisolid cefdinir and solvent and converting semisolid cefdinir to crystalline cefdinir trihemihydrate.

30. A process for making crystalline cefdinir trihernihydrate comprising making a mixture of semisolid cefdinir and solvent and adding water to said mixture.

31. Crystalline cefdinir trihemihydrate prepared by a process comprising making a mixture of semisolid cefdinir and solvent and adding water to said mixture.

32. A process for converting semisolid cefdinir to crystalline cefdinir trihemihydrate comprising making a mixture of cefdinir semisolid and solvent, adding water to said mixture and isolating said crystalline cefdinir trihemihydrate.

33. Crystalline cefdinir trihemihydrate prepared by a process comprising making a mixture of cefdinir semisolid and solvent, adding water to said mixture and isolating said crystalline cefdinir trihemihydrate.

34. A process for converting semisolid cefdinir to crystalline cefdinir trihemihydrate comprising making a mixture of cefdinir semisolid and solvent, adding water to said mixture, centrifugating said mixture and decanting said solvent.

35. Crystalline cefdinir trihemihydrate prepared by a process comprising making a mixture of cefdinir semisolid and solvent, adding water to said mixture, centrifugating said mixture and decanting said solvent.

36. A process for converting semisolid cefdinir to crystalline cefdinir trihemihydrate comprising making a mixture of cefdinir semisolid and solvent, adding water to said mixture, centrifugating said mixture and filtering said mixture under positive pressure.

37. Crystalline cefdinir trihemihydrate prepared by a process comprising making a mixture of cefdinir semisolid and solvent, adding water to said mixture, centrifugating said mixture and filtering said mixture under positive pressure.

38. A process for converting cefdinir trihemihydrate, with or without surface water, to a Cefdinir Crystalline Form A by providing a mixture comprising cefdinir trihemihydrate and an alcohol in which said cefdinir trihemihydrate completely dissolves, causing Cefdinir Crystalline Form A to exist in said mixture, and isolating said Cefdinir Crystalline Form A.

39. Cefdinir Crystalline Form A prepared by a process comprising providing a mixture comprising cefdinir trihemihydrate and an alcohol in which said cefdinir trihemihydrate completely dissolves, causing Cefdinir Crystalline Form A to exist in said mixture, and isolating said Cefdinir Crystalline Form A.

40. A process for converting cefdinir.3.73H₂O, to a Cefdinir Crystalline Form A by providing a mixture comprising cefdinir.3.73H₂O and an alcohol in which said cefdinir.3.73H₂O completely dissolves, causing Cefdinir Crystalline Form A to exist in said mixture, and isolating said Cefdinir Crystalline Form A.

41. Cefdinir Crystalline Form A prepared by a process comprising providing a mixture comprising cefdinir.3.73H₂O and an alcohol in which said cefdinir.3.73H₂O completely dissolves, causing Cefdinir Crystalline Form A to exist in said mixture, and isolating said Cefdinir Crystalline Form A.

42. A process for converting cefdinir.3.76H₂O, to a Cefdinir Crystalline Form A by providing a mixture comprising cefdinir.3.76H₂O and an alcohol in which said cefdinir.3.76H₂O completely dissolves, causing Cefdinir Crystalline Form A to exist in said mixture, and isolating said Cefdinir Crystalline Form A.

43. Cefdinir Crystalline Form A prepared by a process comprising providing a mixture comprising cefdinir.3.76H₂O and an alcohol in which said cefdinir.3.76H₂O completely dissolves, causing Cefdinir Crystalline Form A to exist in said mixture, and isolating said Cefdinir Crystalline Form A.

44. A process for converting cefdinir.3.79H₂O, to a Cefdinir Crystalline Form A by providing a mixture comprising cefdinir.3.79H₂O and an alcohol in which said cefdinir.3.79H₂O completely dissolves, causing Cefdinir Crystalline Form A to exist in said mixture, and isolating said Cefdinir Crystalline Form A.

45. Cefdinir Crystalline Form A prepared by a process comprising providing a mixture comprising cefdinir.3.79H₂O and an alcohol in which said cefdinir.3.79H₂O completely dissolves, causing Cefdinir Crystalline Form A to exist in said mixture, and isolating said Cefdinir Crystalline Form A.

46. A process for converting cefdinir.3.81H₂O, to a Cefdinir Crystalline Form A by providing a mixture comprising cefdinir.3.81H₂O and an alcohol in which said cefdinir.3.81H₂O completely dissolves, causing Cefdinir Crystalline Form A to exist in said mixture, and isolating said Cefdinir Crystalline Form A.

47. Cefdinir Crystalline Form A prepared by a process comprising providing a mixture comprising cefdinir.3.81H₂O and an alcohol in which said cefdinir.3.81H₂O completely dissolves, causing Cefdinir Crystalline Form A to exist in said mixture, and isolating said Cefdinir Crystalline Form A.

48. Cefdinir Crystalline Form A prepared by a process by converting Cefdinir trihemihydrate to Cefdinir Crystalline Form A.

49. Cefdinir Crystalline Form A prepared by a process by converting Cefdinir trihemihydrate to Cefdinir Crystalline Form A, and isolating said Cefdinir Crystalline Form A.
